(19) United States
(12) Patent Application Publication
(10) Pub. No.: US 2021/0052597 A1
(43) Pub. Date: Feb. 25, 2021

(54) HETEROCYCLIC COMPOUNDS USEFUL AS ANTIBACTERIAL AGENTS

(71) Applicant: CADILA HEALTHCARE LIMITED, Ahmedabad, Gujarat (IN)

(72) Inventors: Rajiv SHARMA, Ahmedabad, Gujarat (IN); Pravin IYER, Ahmedabad, Gujarat (IN); Jigar DESAI, Ahmedabad, Gujarat (IN); Sanjay S. KUMAR, Ahmedabad. Gujarat (IN)

(21) Appl. No.: 16/965,475

(22) PCT Filed: Jan. 28, 2019
(86) PCT No.: PCT/IB2019/050668

§ 371 Date: Jul. 28, 2020

(30) Foreign Application Priority Data
Jan. 29, 2018 (IN) 201821003232

(51) Int. Cl. A61K 31/5415; C07D 405/14; A61K 31/4709; A61K 31/538; C07D 413/14; C07D 417/14; A61K 31/498; A61P 31/06

(52) U.S. Cl. CPC A61K 31/5415; C07D 405/14; A61K 31/4709; A61P 31/06; C07D 413/14; C07D 417/14; A61K 31/498; A61K 31/538

(57) ABSTRACT

Present invention relates to compounds of formula (1) its enantiomers, diastereomers, racemic mixtures and their pharmaceutically acceptable salts. This invention also directed to antibacterial drug compounds that are capable of treating bacterial infection which are hard to treat with existing drug compounds.

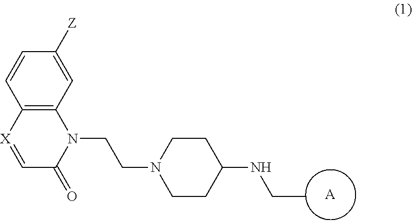

### FIELD OF INVENTION

The present invention relates to antibacterial drug compounds or pharmaceutically acceptable salts, solvates, complexes, hydrates, polymorphs, racemic mixtures, optically active forms and their use for the treatment of diseases or conditions mediated by bacteria. The invention is also directed to antibacterial drug compounds which are capable of treating bacterial infection which are hard to treat with existing drug compounds. Further, the present invention relates to processes of preparing such compounds, their tautomeric forms, novel intermediates involved in their synthesis.

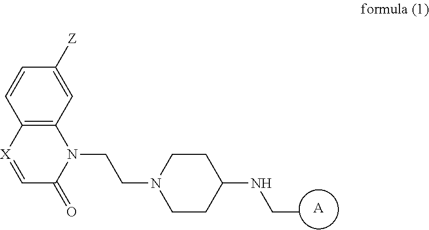

### BACKGROUND TO THE INVENTION

Antibacterial drug resistance is a worldwide issue and the growing rates of antimicrobial resistance in clinical and non-clinical setting possess significant threat to human health globally. Multidrug resistance has become common among some pathogens, Eg. Staphylococcus aureus, Streptococcus pneumonia, Clostridium difficile and Pseudomonas aeruginosa. Of these, Staphylococcus aureus, a Gram +ve bacterium, is a major concern due to its potency and its capacity to adapt to environment condition. Methicillin resistant Staphylococcus aureus (MRSA) is the well-known group of resistant strain and has reached pandemic proportions.

While less widespread, antibiotic resistant Gram −ve strains, such as either Escherichia coli NDM-1 (New Delhi metallo B lactamase 1) or Klebsiella pneumonia NDM-1 are very difficult to treat. Frequently only expensive antibiotics such as vancomycin and colistin are effective against these strains.

Current antibacterial drugs used to treat and prevent bacterial infection have been found to have limited effect. Further, there is a continuing need to identify new compounds with potent antibacterial activity with reduced potential for developing resistance, which possess improved efficacy against bacterial infections that resist treatment with currently available antibiotics, or which possess selectivity against target microorganisms. World Health Organization recognizes antimicrobial resistance as one of the three greatest threats to human health. To address this issue of drug resistance, new chemotype that target critical pathways in bacteria must be developed.

Bacterial type II topoisomerases comprise DNA gyrase and topoisomerase V (TopoIV), which are heterotetrameric enzymes concurrently present in almost all the prokaryotic cells. Both the enzymes are necessary for DNA replication and, hence, for bacterial cell growth and division.

Bacterial type II topoisomerases are proven antibacterial targets, in particular of compounds belonging to fluoroquinolone class. They are broad-spectrum antibacterial drugs that play an important role in treatment of bacterial infections, especially hospital-acquired infections and infections in which resistance to other classes of antibacterial drugs is suspected. Fluoroquinolones act by inhibiting the DNA gyrase and the topoisomerase IV. However, resistance to fluoroquinolones emerged in recent years due to mutations that altered either the active site of the drug targets DNA gyrase and topoisomerase IV or the drug accumulation. In addition, resistance to quinolones can be mediated by plasmids that produce the Qnr protein, which protects the quinolone targets from inhibition (G. A. Jacoby, CID, 2005:41, Suppl. 2, SD120-S126). Regardless of the long term therapeutic success of quinolones in antibacterial chemotherapy, new forms of quinolones-induced resistance in bacterial pathogens are constantly arising indicating a decreased or even complete ineffectiveness of these drugs in antibacterial treatments.

Novel bacterial topoisomerase inhibitor (NBTI) represents emerging class of non-quinolone DNA gyrase and topoisomerase IV inhibitor. NBTI molecules bind to a site that is distinct from, but adjacent to, the catalytic center of DNA gyrase/topoisomerase IV, which is occupied by the quinolones (J Antimicrob Chemother 71:1905-1913, 2016). Hence, NBTI compounds retain potency against fluoroquinolone-resistant (FQR) isolates. NBTIs have been shown to bind to type II topoisomerases during a different phase of the catalytic cycle from fluoroquinolones. Both bind through interactions to the protein-DNA complex, but in contrast to fluoroquinolones that bind to the enzymes with its catalytic tyrosines engaged with the broken double-stranded DNA, NBTIs bind to the enzymes in the presence of intact, unbroken DNA. Numerous efforts has been put by the medicinal chemists for more than a decade to develop NBTI class of antibacterial agent with good invitro/invivo potency and clean toxicity profile, But till date no candidate from NBTI class entered in the market. Numbers of progressive NBTIs were discontinued from clinical trials due to high cardiotoxicity potential denoted as hERG toxicity. Only one candidate (Gepotidacin) is in Phase II clinical trial for the treatment of ABSSSI and Gonorohiae.

Researcher from Vitas pharma (WO 2018172925) reported example VT-03-00065 with 3-nitro-4-methyl phenyl tail piece. Compound showed potent activity against Gram +ve, S. aureus-ATCC 29213 (MIC: 0.25 μg/ml) but was moderately active against S. pnuemoniae ATCC6301 (MIC: 4 μg/ml) and ineffective against Gram −ve strains selected from Enterococcus Faecalis ATCC29212, Moraxella catarrhalis ATCC8176, E. coli ATCC 25922, K pneumonia ATCC 700603 with MIC>32 μg/ml in each strains.

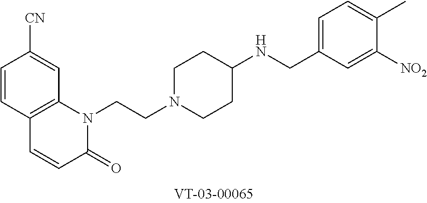 [SMILES: Cc1ccc(CNC2CCN(CCn3c(=O)ccc4ccc(C#N)cc43)CC2)cc1[N+](=O)[O-]]

In the same patent application two more examples VT-03-00042 & VT-03-00043 with bicyclic tail piece were reported. Both these compounds were ineffective against S. aureus-ATCC 29231, MRSA ATCC-33591 and E. coli ATCC 25922 with MIC values of ≥16 μg/ml in all three strains.

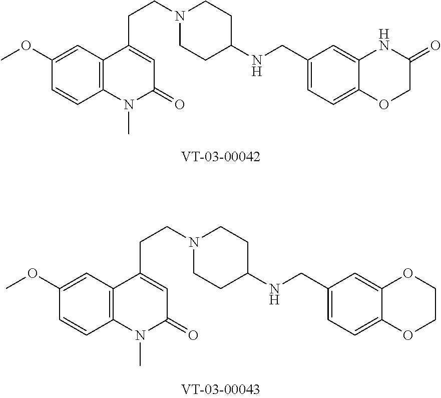 [SMILES: COc1ccc2c(c1)c(CCN1CCC(NCc3ccc4c(c3)NC(=O)CO4)CC1)cc(=O)n2C ; COc1ccc2c(c1)c(CCN1CCC(NCc3ccc4c(c3)OCCO4)CC1)cc(=O)n2C]

Researchers from Glaxo reported compound 15A in patent application WO 2008009700. Example 54 showed MIC values of 1 μM against Mycobacterium tuberculosis. (Journal of Medicinal Chemistry 2014, 57, 4889-4905)

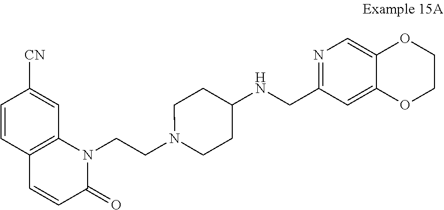 [SMILES: N#Cc1ccc2ccc(=O)n(CCN3CCC(NCc4cc5c(cn4)OCCO5)CC3)c2c1]

Extensive efforts are made to discover novel NBTI class of antibacterial agents, but till date none of them entered in the market. Hence there is an urgent need to develop novel, potent, safe and cost effective antibacterial agent, which will work against broad spectrum and difficult to treat Gram +ve and Gram −ve pathogens.

We herein disclose novel broad spectrum antibacterial compounds of formula (1) which are active against various Gram +ve and Gram −ve strains and are useful in the treatment of diseases or the condition developed due to infection cause by Gram +ve or Gram −ve bacteria.

### SUMMARY OF THE INVENTION

The invention provides compounds that are active against Gram +ve and Gram −ve pathogens and their use for the treatment of infections. The novel compounds are defined by the general formula (1) as given below. The compounds of the present invention are useful in the treatment of the human or animal body, by regulation of pathogens. The compounds of this invention are therefore suitable for the treatment/mitigation/regulation or prophylaxis of number infectious diseases.

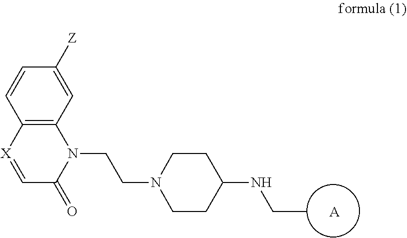

### PREFERRED EMBODIMENTS

The main objective of the present invention is to provide novel compounds of general formula (1), their tautomeric forms, novel intermediates involved in their synthesis, their pharmaceutically acceptable salts, their pharmaceutically acceptable solvates and pharmaceutical compositions containing them or their mixtures suitable for the treatment infectious diseases.

In an embodiment is provided a process for the preparation of novel compounds of general formula (1), their tautomeric forms, enantiomers, diastereomers, racemic mixture or isotopic variants, novel intermediates involved in their synthesis, their pharmaceutically acceptable salts, pharmaceutically acceptable solvates and pharmaceutical compositions containing them.

In another embodiment is provided pharmaceutical compositions comprising compounds of general formula (1), their tautomeric forms, enantiomers, diastereomers, racemic mixture or an isotopic variants, their pharmaceutically acceptable salts, solvates and their mixtures having pharmaceutically acceptable carriers, solvents, diluents, excipients and other media normally employed in their manufacture.

In a further another embodiment is provided the use of the novel compounds of the present invention for the treatment of infectious diseases, by administering a therapeutically effective & non-toxic amount of the compound of formula (1), or their pharmaceutically acceptable compositions to the mammals.

In still further embodiment is provided a use of compounds of formula (1) suitable for the treatment or prevention from bacterial infection.

In another embodiment is provided the compound of formula (1) in combination with one or more suitable pharmaceutically active agent.

### DETAILED DESCRIPTION

Accordingly, the present invention relates to compounds of the general formula (1),

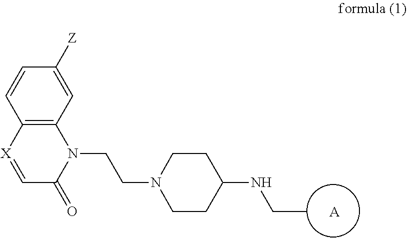

their tautomeric forms, enantiomers, diastereomers, racemic mixture or an isotopic variants, or a pharmaceutically acceptable salt, solvate, or prodrug thereof

### Wherein

Z is selected from CN or F
X is selected from CH or N, provided that whenever Z is F, X is CH.
A is optionally substituted heterocycle selected from

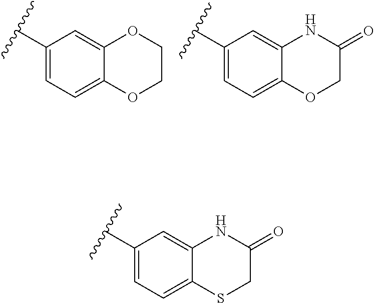

Particularly useful compounds may be selected from:
1-(2-(4-(((2,3-dihydrobenzo[b][1,4]dioxin-6-yl)methyl)amino)piperidin-1-yl)ethyl)-2-oxo-1,2-dihydroquinoline-7-carbonitrile;
2-oxo-1-(2-(4-(((3-oxo-3,4-dihydro-2H-benzo[b][1,4]oxazin-6-yl)methyl)amino)piperidin-1-yl)ethyl)-1,2-dihydroquinoline-7-carbonitrile;
2-oxo-1-(2-(4-(((3-oxo-3,4-dihydro-2H-benzo[b][1,4]thiazin-6-yl)methyl)amino)piperidin-1-yl)ethyl)-1,2-dihydroquinoline-7-carbonitrile;
6-(((1-(2-(7-fluoro-2-oxoquinolin-1(2H)-yl)ethyl)piperidin-4-yl)amino)methyl)-2H-benzo[b][1,4]oxazin-3(4H)-one;
6-(((1-(2-(7-fluoro-2-oxoquinolin-1(2H)-yl)ethyl)piperidin-4-yl)amino)methyl)-2H-benzo[b][1,4]thiazin-3(4H)-one;
4-(2-(4-(((2,3-dihydrobenzo[b][1,4]dioxin-6-yl)methyl)amino)piperidin-1-yl)ethyl)-3-oxo-3,4-dihydroquinoxaline-6-carbonitrile;
3-oxo-4-(2-(4-(((3-oxo-3,4-dihydro-2H-benzo[b][1,4]oxazin-6 yl)methyl)amino)piperidin-1-yl)ethyl)-3,4-dihydroquinoxaline-6-carbonitrile;
3-oxo-4-(2-(4-(((3-oxo-3,4-dihydro-2H-benzo[b][1,4]thiazin-6-yl)methyl)amino)piperidin-1-yl)ethyl)-3,4-dihydroquinoxaline-6-carbonitrile.
1-(2-(4-(((2,3-dihydrobenzo[b][1,4]dioxin-6-yl)methyl)amino)piperidin-1-yl)ethyl)-7-fluoroquinolin-2(1H)-one

Suitable groups and substituents on the groups may be selected from those described anywhere in the specification.
The term “isotopic variant” refers to a compound that contains an unnatural proportion of an isotope at one or more of the atoms that constitute such a compound. In certain embodiments, an “isotopic variant” of a compound contains unnatural proportions of one or more isotopes, including, but not limited to, hydrogen (¹H), deuterium (²H), tritium (³H), carbon-11 (¹¹C), carbon-12 (¹²C), carbon-13 (¹³C) carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O) oxygen-17 (¹⁷O) oxygen-18 (¹⁸O) fluorine-17 (¹⁷F), fluorine-18 (¹⁸F), phosphorus-31 (³¹P), phosphorus-32 (³²P), phosphorus-33 (³³P), sulfur-33 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷C), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I) In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (¹H) deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F) phosphorus-31 (³¹P), sulfur-32 (³²S), sulfur-33 (³³S), sulfur-34 (³⁴S), sulfur-36 (³⁶S) chlorine-35 (³⁵Cl), chlorine-37 (³⁷Cl), bromine-79 (⁷⁹Br), bromine-81 (⁸¹Br), and iodine-127 (¹²⁷I) In certain embodiments, an “isotopic variant” of a compound is in an unstable form, that is, radioactive. In certain embodiments, an “isotopic variant” of a compound contains unnatural proportions of one or more isotopes, including, but not limited to, tritium (³H), carbon-11 (¹¹C), carbon-14 (¹⁴C), nitrogen-13 (¹³N) oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I). It would be understood that, in a compound as provided herein, any hydrogen can be ²H, for example, or any carbon can be ¹³C, as example, or any nitrogen can be N, as example, and any oxygen can be ¹⁸O, where feasible according to the judgment of one of skill. In certain embodiments, an “isotopic variant” of a compound contains unnatural proportions of deuterium.
Compounds of formula (1) may contain one or more asymmetric centers and can thus occur as racemates and racemic mixtures, single enantiomers, diastereomeric mixtures and individual diastereomers. The present invention is meant to comprehend all such isomeric forms of the compounds of formula (1), either as single species or mixtures thereof.
Some of the compounds described herein contain olefinic double bonds, and unless specified otherwise, are meant to include both E and Z geometric isomers.
Some of the compounds described herein may exist with different points of attachment of hydrogen, referred to as tautomers. Such an example may be a ketone and its enol form known as keto-enol tautomers. The individual tautomers as well as mixture thereof are encompassed with compounds of formula (1).

### List of Abbreviation

### ACN: Acetonitrile

DMF: Dimethyl formamide

### DCM: Dichloromethane

### EDC: Dichloroethane

### EtOH: Ethanol

TFA: Trifluoro acetic acid

### THF: Tetrahydrofurane

DIPEA: Diisopropyl ethyl amine
EtOAc: Ethyl acetate

### h: Hour(s)

HCl: Hydrochloric acid

### min: Minute(s)

### MeOH: Methanol

NaBH₃CN: Sodium cyanoborohydride
NaB(OAc)₃H: Sodium triacetoxyborohydride
NaBH₄: Sodium borohydride
rt or RT: room temperature (25-30° C.)
t_{Ret}: Retention time
Cs₂CO₃: Cesium carbonate
TEA: Triethyl amine

### Instrument Details

### Mass Spectrum Recorded on LC-MS 2010-A Shimadzu.

### NMR Spectrum Recorded on Bruker Avanc 400 MHz

The compounds of the present invention may be prepared using the methods described below, together with conventional techniques known to those skilled in the art of organic synthesis or variation thereon as appreciated by those skilled in the art. Preferred methods include, but are not limited to those described below, where all symbols are as defined earlier

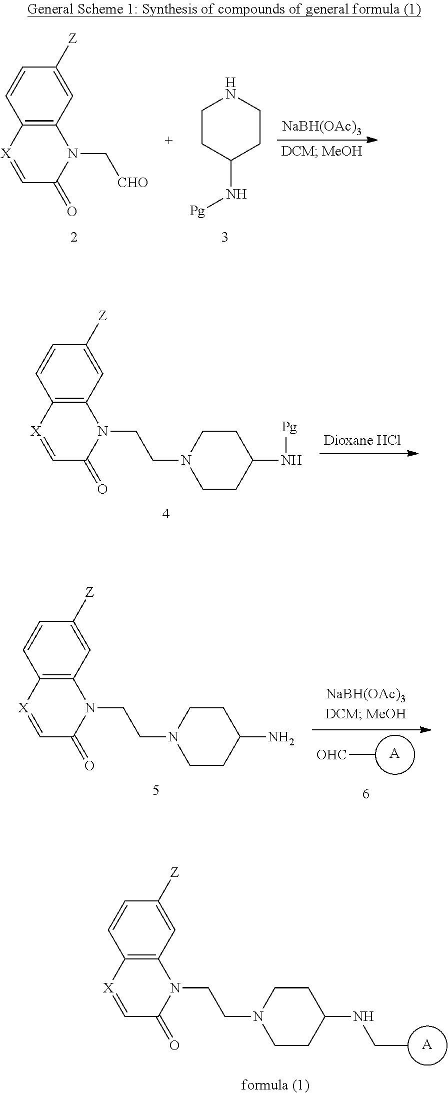

Aldehyde derivative (2) on reductive amination with 4-amino protected piperidine (3) in the presence of suitable reducing agents selected from NaBH₃CN, NaB(OAc)₃H, NaBH₄ in solvent selected from DCM, ACN, MeOH, EtOH and combination thereof afforded compound of formula (4). Deprotection of Boc in (4) with Dioxane.HCl or TFA/DCM resulted in amine compound (5). Amine compound of formula (5) on reductive amination with suitable aldehyde derivative (6) in presence NaBH₃CN, NaB(OAc)₃H, NaBH₄ in solvents selected from DCM, ACN, MeOH, EtOH and combination thereof, afforded compound of formula (1)

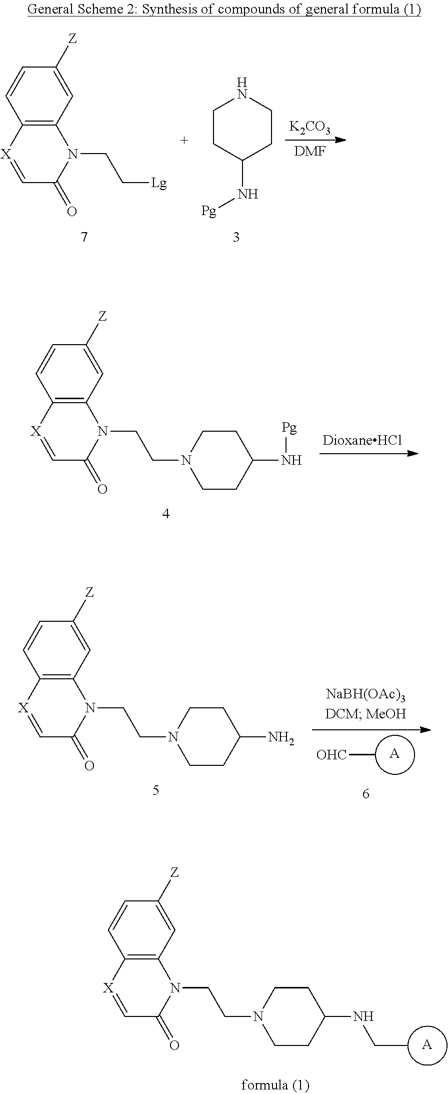

Alternatively compound of formula (1) can be prepared by reacting compound of formula (7) (Where Lg is leaving group selected from Cl, Br, I, OMs) with 4-amino protected piperidine (3) (Where Pg is amine protecting group selected from Boc, CBz, Pivalyl) in the presence of base selected from K₂CO₃, CS₂CO₃, TEA, DIEA, DBU in solvent like DCM, EDC, DMF, ACN afforded compound of formula (4). Deprotection of Pg group in (4) using dioxane. HCl, TFA, HCl in solvent selected from DCM, EDC afforded amine derivative of formula (5). Amine compound of formula (5) on reductive amination with suitable aldehyde derivative (6) in presence NaBH₃CN, NaB(OAc)₃H, NaBH₄ in solvents selected from DCM, ACN, MeOH, EtOH and combination thereof, afforded compound of formula (1).

The compound of formula (2) and (7) can be synthesized as per the reference WO 2006023467 and Journal of Medicinal chemistry 55(15), 6916, 2012. Compound of formula (3) was used directly from commercial source.

The pharmaceutically acceptable salts forming a part of this invention may be prepared by treating the compound of formula (1) with suitable acids in suitable solvents by processes known in the art.

The invention is further exemplified by the following examples below, which provides some of the several preferred embodiments of the present invention. These examples are provided merely as representative embodiments and should not be construed to limit the scope of the invention in any way.

### Synthesis of Intermediates

| No | Structure | Chemical Name |
|---|---|---|
| A1 | 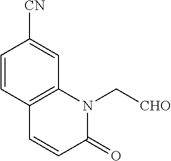 [SMILES: N#Cc1ccc2ccc(=O)n(CC=O)c2c1] | 2-oxo-1-(2-oxoethyl)-1,2- dihydroquinoline-7- carbonitrile |
| A2 | 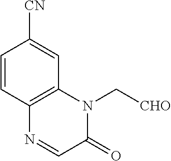 [SMILES: N#Cc1ccc2ncc(=O)n(CC=O)c2c1] | 3-oxo-4-(2-oxoethyl)-3,4- dihydroquinoxaline-6- carbonitrile |
| A3 | 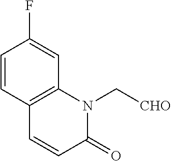 [SMILES: O=CCn1c(=O)ccc2ccc(F)cc21] | 2-(7-fluoro-2- oxoquinolin-1(2H)- yl)acetaldehyde |

### Preparation of Intermediate:

Intermediate A1: 2-Oxo-1-(2-oxoethyl)-1,2-dihydroquinoline-7-carbonitrile was prepared according to the procedure reported in Journal of Medicinal chemistry 2012, 55, 6916-6933.

### Intermediate A2:

### Step 1: Preparation of 3-oxo-3,4-dihydroquinoxaline-6-carbonitrile

3-oxo-3,4-dihydroquinoxaline-6-carbonitrile was prepared as per the procedure reported in WO 2014024056.

### Step II: Preparation of 4-allyl-3-oxo-3,4-dihydroquinoxaline-6-carbonitrile

Placed 3-oxo-3,4-dihydroquinoxaline-6-carbonitrile (650 mg, 3.80 mmol) and DMF (6.5 ml) in rb flask at 25° C. Mixture was cooled to 0-5° C. and NaH (401 mg, 8.35 mmol) was added. Reaction mixture was stirred at 25° C. for 10 min and 3-iodoprop-1-ene (0.764 ml, 8.35 mmol) was added at 0-5° C. The reaction mixture was stirred at 25° C. for 10 min. After completion of reaction, the reaction mixture was diluted with water (50 ml) and the aqueous layer was extracted with EtOAc (50 ml)×4. Combined the organic layers and washed with water and brine solution. The organic layer was dried over sodium sulfate filtered and concentrated under reduced pressure to afford crude product which was purified using Flash Column Chromatography. Column: 12 gm Redi Sep Column & mobile phase: 10% EtOAc in Hexane

### Step III: Preparation of 3-oxo-4-(2-oxoethyl)-3,4-dihydroquinoxaline-6-carbonitrile

Placed 4-allyl-3-oxo-3,4-dihydroquinoxaline-6-carbonitrile (350 mg, 1.657 mmol), dioxane (35 ml) and water (11 mL) to round bottom flask. To this, sodium periodate (1170 mg, 5.47 mmol) was added followed by osmium(VIII) oxide (2.106 ml, 0.331 mmol). Reaction mixture was stirred for 4 hr at 25° C. After completion of reaction, Mixture was quenched in water (25 ml). Compound was extracted by adding ethyl acetate (20 ml*3). Combined organic layers was washed with water and evaporated under reduced pressure to get crude compound. The title compound was purified by flash column chromatography using 2% Methanol in DCM in as mobile phase. [150 mg, 42%]

Intermediate A3: 2-(7-Fluoro-2-oxoquinolin-1(2H)-yl)acetaldehyde was prepared according to the procedure reported in WO 2008009700

### Synthesis of Intermediates

| No | Structure | Chemical Name |
|---|---|---|
| B1 | 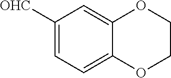 [SMILES: O=Cc1ccc2c(c1)OCCO2] | 2,3-dihydrobenzo[b][1,4] dioxine-6-carbaldehyde |
| B2 | 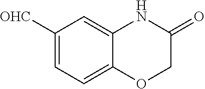 [SMILES: O=Cc1ccc2c(c1)NC(=O)CO2] | 3-oxo-3,4-dihydro- 2H-benzo[b][1,4] oxazine-6-carbaldehyde |
| B3 | 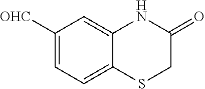 [SMILES: O=Cc1ccc2c(c1)NC(=O)CS2] | 3-oxo-3,4-dihydro-2H- benzo[b][1,4]thiazine- 6-carbaldehyde |

Intermediate B1: 2,3-dihydrobenzo[b][1,4]dioxine-6-carbaldehyde was prepared as per the process reported in WO 2012049555.
Intermediate B2: 3-oxo-3,4-dihydro-2H-benzo[b][1,4]oxazine-6-carbaldehyde was prepared as per the procedure reported in WO 2010111626.
Intermediate B3: 3-oxo-3,4-dihydro-2H-benzo[b][1,4]thiazine-6-carbaldehyde prepared as per the procedure reported in WO 2014057415

In one of the preferred embodiment is provided novel compounds of the present invention for the treatment of infectious diseases caused by various bacterial strains such as Staphylococcus aureus, Staphylococcus pneumonia, Enterococcus faecalis, Escherichia coli, Acinetobacter baumannii, Klebsiella pneumonia or Mycobacterium tuberculosis.

In another embodiment is provided compound of formula (1) in combination with one or more pharmaceutically active agent selected from amphenicol, β-lactum, a tetracycline, an aminoglycoside, a quinolone, a motilin, a macrolide, an azole, a non-steroidal anti inflammatory drug (NSAID), a glucocorticosteroid class of compounds or their pharmaceutically acceptable salts

### Example 1: Preparation of 1-(2-(4-(((2,3-dihydrobenzo[b][1,4]dioxin-6-yl)methyl) amino)piperidin-1-yl)ethyl)-2-oxo-1,2-dihydroquinoline-7-carbonitrile

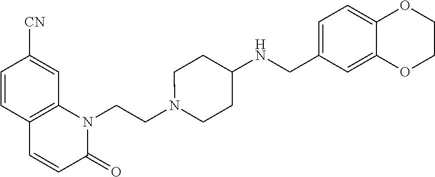 [SMILES: N#Cc1ccc2ccc(=O)n(CCN3CCC(NCc4ccc5c(c4)OCCO5)CC3)c2c1]

### Step 1: Synthesis of tert-butyl (1-(2-(7-cyano-2-oxoquinolin-1(2H)-yl) ethyl) piperidin-4-yl)carbamate

To a stirred solution of 2-oxo-1-(2-oxoethyl)-1,2-dihydroquinoline-7-carbonitrile (0.780 g, 3.68 mmol) in THF (30 ml) was added tert-butyl piperidin-4-ylcarbamate (0.811 g, 4.05 mmol) at 25° C. The reaction mixture was refluxed for 2 hr. Mixture was cooled to 100 C and sodium triacetoxy borohydride (2.339 g, 11.04) mmol was added. The mixture was stirred at room temperature for 2 hrs, methanol (12 ml) was added to reaction mixture and was then stirred for 16 hr. After completion of reaction, mixture was quenched in water; compound was extracted with ethyl acetate (25 ml*2). Combined organic layers was washed with water, dried over sodium sulfate and evaporated under reduced pressure to afford crude mass which was purified by silica gel column chromatography using ethyl acetate:hexane (50:50) mobile phase. The title compound obtained as off white solid. (1.0 gm, 23%)

### Step 2: Synthesis of 1-(2-(4-aminopiperidin-1-yl)ethyl)-2-oxo-1,2-dihydroquinoline-7-carbonitrile

Placed tert-butyl (1-(2-(7-cyano-2-oxoquinolin-1(2H)-yl)ethyl)piperidin-4-yl)carbamate (1 g, 2.52 mmol) in rb flask followed by DCM (80 ml). To this, TFA (6.08 ml, 79 mmol) was added and mixture was stirred for 16 hrs at room temperature. After completion of reaction, organic volatiles were removed under reduced pressure. Crude product obtained was used directly for the next reaction. (0.4 gm 53.5%)

### Step 3: Synthesis of 1-(2-(4-(((2,3-dihydrobenzo[b][1,4]dioxin-6-yl)methyl) amino)piperidin-1-yl)ethyl)-2-oxo-1,2-dihydroquinoline-7-carbonitrile

In a round-bottomed flask was added 1-(2-(4-aminopiperidin-1-yl)ethyl)-2-oxo-1,2-dihydroquinoline-7-carbonitrile (0.250 g, 0.844 mmol) and THF (40 ml). To this, 2,3-dihydrobenzo(b)(1,4)dioxine-6-carbaldehyde (0.126 g, 0.765 mmol) was added and mixture was heated at 75° C. for 2 hr. Mixture was cooled to room temperature and sodium triacetoxy borohydride (0.487 g, 2.298 mmol) was added. Mixture was stirred at room temperature for 2 hr. After completion of reaction, mixture was quenched in water. Compound was extracted with ethyl acetate (25 ml*2). Combined organic layer was dried over sodium sulfate, evaporated under reduced pressure to give crude product. The title compound was purified by silica gel column chromatography using DCM: MeOH as mobile phase. (180 mg, 17%). ¹H NMR (DMSO d₆): 8.08 (1H, s), 8.00 (1H, d, J=8.0 Hz), 7.91 (1H, d, J=8), 7.66 (1H, d, J=1.2 Hz), 6.83 (1H, s), 6.79-6.77 (3H, m), 4.37 (2H, t, J=8 Hz), 4.10 (4H, s), 3.60 (2H, s), 2.91-2.88 (2H, m), 2.09-2.03 (2H, m), 1.98 (3H, s), 1.90-1.77 (2H, m), 1.23-1.19 (2H, m). ESI-MS: 445.15 (M+H)⁺

### Example 2: Preparation of 2-oxo-1-(2-(4-(((3-oxo-3,4-dihydro-2H-benzo[b][1,4]oxazin-6-yl)methyl)amino)piperidin-1-yl)ethyl)-1,2-dihydroquinoline-7-carbonitrile

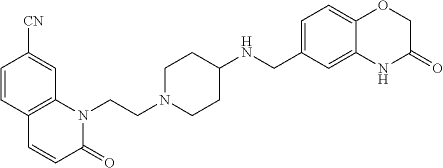 [SMILES: N#Cc1ccc2ccc(=O)n(CCN3CCC(NCc4ccc5c(c4)NC(=O)CO5)CC3)c2c1]

1-(2-(4-(((3,4-dihydro-2H-pyrano[2,3-c]pyridin-6-yl)methyl)amino)piperidin-1-yl)ethyl)-2-oxo-1,2-dihydroquinoline-7-carbonitrile was prepared similar to the procedure described in Example 1 but using 3-oxo-3,4-dihydro-2H-benzo[b][1,4]oxazine-6-carbaldehyde B2 as starting material in step III. The title compound was characterized by spectral analysis. ¹H NMR (DMSO d₆): 10.67 (1H, s), 8.08 (1H, s), 8.00 (1H, d, J=9.6 Hz), 7.91 (1H, d, J=8 Hz), 7.66-7.64 (1H, m), 6.89-6.87 (3H, m), 6.78 (1H, d, J=9.6), 4.52 (2H, s), 4.36 (2H, d, J=6.8), 3.64 (2H, s), 3.17 (2H, s), 2.93-2.90 (2H, m), 2.02 (2H, t, J=10.4 Hz), 1.80-1.77 (2H, m), 1.26-1.23 (3H, m). ESI-MS: 458.01 (M)⁺

### Example 3: Preparation of 2-oxo-1-(2-(4-(((3-oxo-3,4-dihydro-2H-benzo[b][1,4]thiazin-6-yl)methyl)amino)piperidin-1-yl)ethyl)-1,2-dihydroquinoline-7-carbonitrile

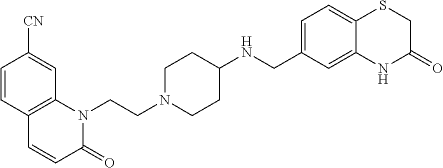 [SMILES: N#Cc1ccc2ccc(=O)n(CCN3CCC(NCc4ccc5c(c4)NC(=O)CS5)CC3)c2c1]

2-oxo-1-(2-(4-(((3-oxo-3,4-dihydro-2H-benzo[b][1,4]thiazin-6-yl)methyl)amino) piperidin-1-yl)ethyl)-1,2-dihydroquinoline-7-carbonitrile was prepared similar to the procedure described in Example 1 but using 3-oxo-3,4-dihydro-2H-benzo[b][1,4]thiazine-6-carbaldehyde B3 as starting material in step III. The title compound was characterized by spectral analysis. ¹H NMR (DMSO d₆): 10.52 (1H, s), 8.08 (1H, s), 8.00 (1H, d, J=9.6 Hz), 7.91 (1H, d, J=8.0 Hz), 7.65 (1H, dd, J1=1.2 Hz, J2=8.0 Hz), 7.25-7.23 (1H, m), 6.97-6.94 (2H, m), 6.78 (1H, d, J=9.2 Hz), 4.37 (2H, t, J=6.8 Hz), 3.70-3.60 (2H, m), 3.43 (2H, s), 2.93-2.91 (2H, m), 2.68-2.67 (1H, m), 2.04-1.99 (2H, m), 1.80-1.77 (2H, m), 1.26-1.23 (2H, m). ESI-MS: 473 (M)⁺

### Example 4: Preparation of 6-(((1-(2-(7-fluoro-2-oxoquinolin-1(2H)-yl) ethyl)piperidin-4-yl)amino)methyl)-2H-benzo[b][1,4]oxazin-3(4H)-one

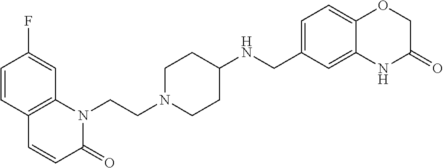 [SMILES: O=C1COc2ccc(CNC3CCN(CCn4c(=O)ccc5ccc(F)cc54)CC3)cc2N1]

6-(((1-(2-(7-fluoro-2-oxoquinolin-1(2H)-yl)ethyl)piperidin-4-yl)amino)methyl)-2H-benzo[b][1,4]oxazin-3(4H)-one was prepared similar to the procedure described in Example 1 but using 2-(7-fluoro-2-oxoquinolin-1(2H)-yl)acetaldehyde A3 in step I and 3-oxo-3,4-dihydro-2H-benzo[b][1,4]oxazine-6-carbaldehyde B2 as starting material in step III. The title compound was characterized by spectral analysis. ¹H NMR (DMSO d₆): 10.67 (1H, s), 7.91 (1H, d, J=8.4 Hz), 7.90-7.77 (1H, m), 7.40 (1H, dd, J1=2.0 Hz, J2=12.0 Hz), 7.16-7.11 (1H, m), 6.91-6.88 (3H, m), 6.56 (1H, d, J=9.2 Hz), 4.53 (2H, s), 4.31 (2H, t, J=6.8 Hz), 3.68 (2H, s), 2.94-2.92 (2H, m), 2.05-1.99 (2H, m), 1.20-1.79 (2H, m), 1.28-1.26 (2H, m). ESI-MS: 451.20 (M+H)]⁺.

### Example 5: Preparation of 6-(((1-(2-(7-fluoro-2-oxoquinolin-1(2H)-yl)ethyl)piperidin-4-yl)amino)methyl)-2H-benzo[b][1,4]thiazin-3(4H)-one

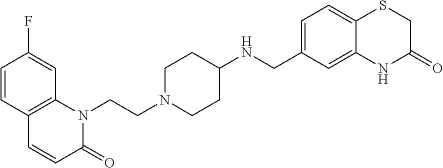 [SMILES: O=C1CSc2ccc(CNC3CCN(CCn4c(=O)ccc5ccc(F)cc54)CC3)cc2N1]

6-(((1-(2-(7-fluoro-2-oxoquinolin-1(2H)-yl)ethyl)piperidin-4-yl)amino)methyl)-2H-benzo[b][1,4]thiazin-3(4H)-one was prepared similar to the procedure described in Example 1 but using 2-(7-fluoro-2-oxoquinolin-1(2H)-yl)acetaldehyde A3 in step I and 3-oxo-3,4-dihydro-2H-benzo[b][1,4]thiazine-6-carbaldehyde B3 as starting material in step III. The title compound was characterized by spectral analysis. ¹H NMR (DMSO d₆): 10.49 (1H, s), 7.91 (1H, d, J=9.6 Hz), 7.79 (1H, dd, J1=6.8 Hz, J2=8.8 Hz), 740 (1H, dd, J1=2.0 Hz, J2=12.0 Hz), 7.30-7.20 (1H, m), 7.14 (1H, d, J=2.0 Hz), 7.00-6.96 (2H, m), 6.56 (1H, d, J=9.6 Hz), 4.12 (2H, t, J=6.8 Hz), 3.43 (2H, s), 3.31 (2H, s), 2.95-2.89 (2H, m), 2.45-2.40 (1H, m), 2.03-1.99 (2H, m), 1.95-1.91 (2H, m), 1.20-1.16 (2H, m). ESI-MS: 467.17 (M+H)⁺.

### Example 6 3-oxo-4-(2-(4-(((3-oxo-3,4-dihydro-2H-benzo[b][1,4]thiazin-6-yl)methyl)amino)piperidin-1-yl)ethyl)-3,4-dihydroquinoxaline-6-carbonitrile

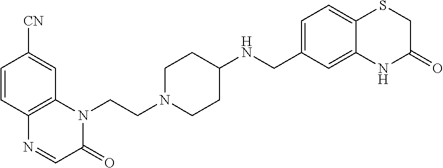 [SMILES: N#Cc1ccc2ncc(=O)n(CCN3CCC(NCc4ccc5c(c4)NC(=O)CS5)CC3)c2c1]

3-oxo-4-(2-(4-(((3-oxo-3,4-dihydro-2H-benzo[b][1,4]thiazin-6-yl)methyl)amino) piperidin-1-yl)ethyl)-3,4-dihydroquinoxaline-6-carbonitrile was prepared similar to the procedure described in Example 1 but using 3-oxo-4-(2-oxoethyl)-3,4-dihydroquinoxaline-6-carbonitrile A2 in step I and 3-oxo-3,4-dihydro-2H-benzo[b][1,4]thiazine-6-carbaldehyde B3 as starting material in step III. The title compound was characterized by spectral analysis. ¹H NMR (DMSO d₆): 10.50 (1H, s), 8.38 (1H, s), 8.21 (1H, d, J=1.2 Hz), 7.98 (1H, d, J=8.4 Hz), 7.78 (1H, dd, J1=1.6 Hz, J2=8.4 Hz), 7.23 (1H, d, J=7.6 Hz), 6.62-6.35 (2H, m), 4.33 (2H, t, J=6.4 Hz), 3.60-3.55 (2H, m), 3.42 (2H, s), 2.90-2.88 (2H, m), 2.04-1.99 (3H, m), 1.77-1.74 (2H, m), 1.24-1.19 (4H, m). ESI-MS: 475.17 (M+H)*.

Following examples can be prepared in similar manner by using method mentioned above.

### Example 7: 3-oxo-4-(2-(4-(((3-oxo-3,4-dihydro-2H-benzo[b][1,4]oxazin-6-yl)methyl) amino)piperidin-1-yl)ethyl)-3,4-dihydroquinoxaline-6-carbonitrile

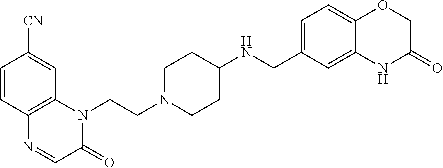 [SMILES: N#Cc1ccc2ncc(=O)n(CCN3CCC(NCc4ccc5c(c4)NC(=O)CO5)CC3)c2c1]

### Example 8: 4-(2-(4-(((2,3-dihydrobenzo[b][1,4]dioxin-6-yl)methyl)amino)piperidin-1-yl)ethyl)-3-oxo-3,4-dihydroquinoxaline-6-carbonitrile

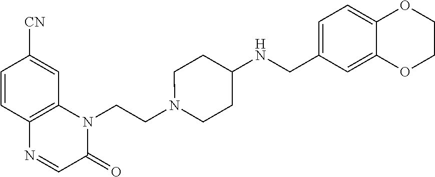 [SMILES: N#Cc1ccc2ncc(=O)n(CCN3CCC(NCc4ccc5c(c4)OCCO5)CC3)c2c1]

### Example 9: 1-(2-(4-(((2,3-dihydrobenzo[b][1,4]dioxin-6-yl)methyl)amino)piperidin-1-yl)ethyl)-7-fluoroquinolin-2(1H)-one

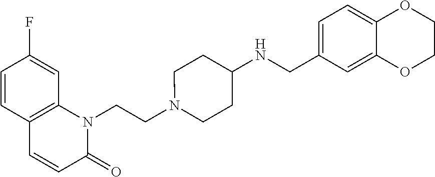 [SMILES: O=c1ccc2ccc(F)cc2n1CCN1CCC(NCc2ccc3c(c2)OCCO3)CC1]

### Antibacterial Activity:

The compounds of Formula (1) are of interest due to their potent antibacterial effects. The ability of the invention compounds disclosed herein to achieve an antibacterial effect may be evaluated with regard to their ability to inhibit the growth of bacterial species like Escherichia coli ATCC 25922, Staphylococcus aureus ATCC 29213 using an assay based on the following Minimum Inhibitory Concentration (MIC) protocol:

The test bacteria are grown in Muellor Hinton Broth (M1657)—MHB, 25 grams of the powder is dissolved in 1000 ml distilled water and sterilized by autoclaving at 15 lbs pressure (121° C.) for 20 minutes. The medium sterility is checked by incubating at 37° C. for a period of 48 h.

Bacterial cultures that are stored as glycerol stocks at −80° C. are sub cultured on LB agar plates to obtain isolated colonies. A single colony of each strain is cultured in LB broth. The cultures are incubated at 37° C., 200 rpm till they reach an optical density (OD at 600 nm) of 0.8 to 1. This log phase culture is diluted in LB broth to a cell number of 5-8*105 CFU/mL to be used as inoculum for MIC experiments.

Test compounds were diluted in their respective solvents and added to a final concentration ranging from 16 to 0.12 μg/ml in 150 μl MHB in 96 well plates.

Controls to monitor the effects of DMSO and media sterility are included. The plates were incubated at 37° C. overnight in a humidified incubator. The following morning, the plates are read using a Spectrophotometer at 600 nM wavelength.

Minimum Inhibitory Concentration (MIC) is defined as the lowest drug concentration containing well which shows no turbidity. The antibacterial activity (MIC) determined against representative Gram positive (S. aureus) and Gram-negative (E. coli) pathogen were reported Table 1.

The exemplified compounds belonging to Formula I demonstrated potent antibacterial activity.

Minimum Inhibitory
Concentration (μg/mL)

| | S. aureus | E. coli |
|---|---|---|
| Example | ATCC 29213 | ATCC 25922 |
| 1 | 0.03 | 0.5 |
| 2 | 0.25 | 0.25 |
| 3 | 0.06 | 0.125 |
| 4 | <0.125 | <0.125 |
| 5 | 0.25 | 0.25 |
| 6 | <0.125 | <0.125 |

### MIC Against Fluoroquinolone Resistant Strain:

Compounds were screened against quinolone resistant clinical strains of Staphylococcus aureus (ZYABL6) and are found to be potent.

| | Minimum Inhibitory |
|---|---|
| | Concentration (μg/mL) |
| | Staphylococcus aureus |
| Example | ZYABL 006 |
| 1 | <0.125 |
| 2 | <0.125 |
| 3 | <0.125 |
| 4 | <0.125 |
| 5 | <0.125 |
| Ciprofloxacin | 16 |

### Target Specificity of Compounds

Evaluation of Topoisomerase II/IV inhibition using gel electrophoresis method: Topoisomerase II/IV (1 unit, Inspiralis) was added to a reaction mixture of DNA (pHOT/kDNA, Topogen) and ATP in appropriate buffers following which different concentrations of the test compounds were added and incubated at 37° C. for 30 min. The reaction was terminated after 30 minutes by the addition of stop buffer. The resulting DNA (relaxed, supercoiled or decatanated) was extracted using chloroform: isoamyl alcohol mixture and was separated using 1% agarose gel electrophoresis. Gel was stained with ethidium bromide for 20 minutes, washed with distilled water and the images were captured for further analysis. The band Intensities from the image were measured using Image J software and the half-maximal minimum inhibitory concentration was deduced using Graphpad Prism.

Invitro IC₅₀

| | Topoisomerase II | Topoisomerase IV |
|---|---|---|
| Example | (nM) | (μM) |
| 1 | 44 | 0.9 |
| 2 | 3.4 | 1.3 |
| 3 | 3.2 | 0.3 |
| 4 | 6.9 | 0.4 |
| 5 | 38.4 | 0.8 |

hERG Binding Study

### FluxOR™ Potassium Ion Channel Assay

Thallium flows down its concentration gradient into the cells, and channel activity is detected with indicator dye that increases in cytosolic fluorescence. Stably expressing hERG CHO cells are checked with test compounds for hERG liability. Compounds incubated for 20 min with cells then stimulation buffer was added and fluorescence measured on TECAN multimode reader. If test compound is inhibiting hERG channel then it will not allow the thallium to flows down in the cells. Vehicle control is considered as total hERG response while treatment with Astemizole as a total hERG channel inhibition and based on that test compounds hERG channel inhibition calculated. The synthesized compounds did not showed significant hERG liability.

| Example | IC₅₀ (μM) |
|---|---|
| 1 | >30 |
| 2 | >10 |
| 3 | >10 |
| 4 | >10 |
| 5 | >10 |

### MIC Testing Against Broad Panel of Strains

The Example 3 was further evaluated for MIC using various Gram +ve and Gram-ve strains, which are resistant to existing antibiotics, as per the protocol reported earlier.

| | MIC (μg/ml) | | | | | |
|---|---|---|---|---|---|---|
| | Staphylococcus | Staphylococcus | Enterococcus | Escherichia | Acinetobacter | Klebsiella |
| | aureus¹ | pneumonia² | faecalis³ | coli⁴ | baumannii⁵ | pneumonia |
| Compound | (BAA38) | (ATCC700906) | (ATCC700802) | (ATCC10536) | (BAA1605) | (ATCC35657) |
| Example 3 | 0.125 | 0.125 | 0.5 | 0.125 | 0.25 | 0.25 |

¹Multidrug - Methicillin, Penicillin, Streptomycin, Tetracycline resistant
²Resistant to erythromycin, penicillin, tetracycline, chloramphenicol
³Resistant to gentamicins and vancomycin Sensitive to daptomycin and streptomycin
⁴Resistant to polymyxin
⁵Resistant to Ceftazidime, Gentamicin, Ticarcillin, Piperacillin, Aztreonam, Cefepime, Ciprofloxacin, Imipenem, and Meropemem. Sensitive to Amikacin and Tobramycin

Mycobacterium tuberculosis H37Rv Inhibition Assay

The measurement of the minimum inhibitory concentration (MIC) for each tested compound was performed in 96 wells flat bottom, polystyrene microtiter plates following standard protocol. Example 3 showed potent inhibition against Mycobacterium tuberculosis.

| Compounds ID | MIC(μM) |
|---|---|
| Example 3 | 0.25 |

1. Compounds of formula (I), 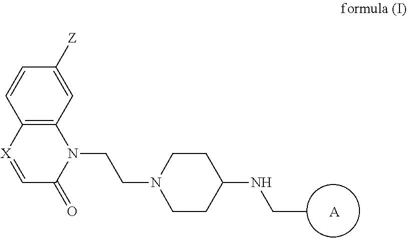
its single enantiomer, a racemic mixture, a mixture of diastereomers, or an isotopic variant thereof;
wherein,
A is optionally substituted heterocycle selected from 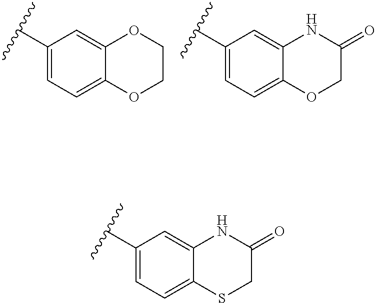
Z selected from CN or F
X selected from CH or N, provided that whenever Z is F, X is CH.

2. Compounds of formula (1) as claimed in claim 1 can be selected from
1-(2-(4-(((2,3-dihydrobenzo[b][1,4]dioxin-6-yl)methyl)amino)piperidin-1-yl)ethyl)-2-oxo-1,2-dihydroquinoline-7-carbonitrile;
2-oxo-1-(2-(4-(((3-oxo-3,4-dihydro-2H-benzo[b][1,4]oxazin-6-yl)methyl)amino)piperidin-1-yl)ethyl)-1,2-dihydroquinoline-7-carbonitrile;
2-oxo-1-(2-(4-(((3-oxo-3,4-dihydro-2H-benzo[b][1,4]thiazin-6-yl)methyl)amino)piperidin-1-yl)ethyl)-1,2-dihydroquinoline-7-carbonitrile;
6-(((1-(2-(7-fluoro-2-oxoquinolin-1(2H)-yl)ethyl)piperidin-4-yl)amino)methyl)-2H-benzo[b][1,4]oxazin-3(4H)-one;
6-(((1-(2-(7-fluoro-2-oxoquinolin-1(2H)-yl)ethyl)piperidin-4-yl)amino)methyl)-2H-benzo[b][1,4]thiazin-3(4H)-one;
4-(2-(4-(((2,3-dihydrobenzo[b][1,4]dioxin-6-yl)methyl)amino)piperidin-1-yl)ethyl)-3-oxo-3,4-dihydroquinoxaline-6-carbonitrile;
3-oxo-4-(2-(4-(((3-oxo-3,4-dihydro-2H-benzo[b][1,4]oxazin-6 yl)methyl)amino)piperidin-1-yl)ethyl)-3,4-dihydroquinoxaline-6-carbonitrile;
3-oxo-4-(2-(4-(((3-oxo-3,4-dihydro-2H-benzo[b][1,4]thiazin-6-yl)methyl)amino)piperidin-1-yl)ethyl)-3,4-dihydroquinoxaline-6-carbonitrile;
1-(2-(4-(((2,3-dihydrobenzo[b][1,4]dioxin-6-yl)methyl)amino)piperidin-1-yl)ethyl)-7-fluoroquinolin-2(1H)-one.

3. A pharmaceutical composition comprising a therapeutically effective and non-toxic amount of a compound of formula (1) as claimed in claim 1 and optionally one or more pharmaceutically acceptable carriers, diluents or excipients.

4. The pharmaceutical composition as claimed in claim 3 provides novel compounds that are useful for treating or preventing bacterial infection caused by Staphylococcus aureus, Staphylococcus pneumonia, Enterococcus faecalis, Escherichia coli, Acinetobacter baumannii, Klebsiella pneumonia or Mycobacterium tuberculosis.

5. The use of compound of formula (1) or its pharmaceutical composition as claimed in claim 1 suitable for the treatment or prevention from bacterial infections caused by Staphylococcus aureus, Staphylococcus pneumonia, Enterococcus faecalis, Escherichia coli, Acinetobacter baumannii, Klebsiella pneumonia or Mycobacterium tuberculosis.

6. A method of treating bacterial infection which comprising administering to a patient in need thereof an effective amount of a compound of Formula (1) as claimed in claim 1 or its suitable pharmaceutical composition.

7. Compound of formula (1) as claimed in claim 1 in combination with one or more pharmaceutically active agent selected from amphenicol, a β-lactum, a tetracycline, an aminoglycoside, a quinolone, a motilin, a macrolide, an azole, a non-steroidal anti-inflammatory drug (NSAID), a glucocorticosteroid class of compounds or their pharmaceutically acceptable salts.
